(19) United States
(12) Patent Application Publication
(10) Pub. No.: US 2023/0196569 A1
(43) Pub. Date: Jun. 22, 2023

(54) CALCIUM ARC OF BLOOD VESSEL WITHIN INTRAVASCULAR IMAGE AND ASSOCIATED SYSTEMS, DEVICES, AND METHODS

(71) Applicant: PHILIPS IMAGE GUIDED THERAPY CORPORATION, SAN DIEGO, CA (US)

(72) Inventors: Asher COHEN, SAN DIEGO, CA (US); Pei-Yin CHAO, EINDHOVEN (NL); Ronald HELMSTRIJD, EINDHOVEN (NL)

(21) Appl. No.: 18/082,482

(22) Filed: Dec. 15, 2022

Related U.S. Application Data
(60) Provisional application No. 63/292,521, filed on Dec. 22, 2021.

(51) Int. Cl. G06T 7/00; G06T 7/64; G06F 3/04883; G06F 3/14

(52) U.S. Cl. CPC G06T 7/0012; G06T 7/64; G06F 3/04883; G06F 3/14; G06T 2207/20101; G06T 2207/30101

(57) ABSTRACT

A system includes a processor circuit in communication with an intraluminal imaging device. The processor circuit is configured to receive an intraluminal image obtained by the intraluminal imaging device while the intraluminal imaging device is positioned within a body lumen of a patient. The processor circuit also receive a user input selecting one location within the intraluminal image and another user input selecting another location within the intraluminal image. These two locations within the intraluminal image define boundaries of a sector of the intraluminal image associated with a tissue type. The processor circuit determines an angle of the sector defined by the two locations. The intraluminal image and a visual representation of the angle are displayed on a screen display.

### CROSS-REFERENCE TO RELATED APPLICATIONS

This application claims priority to and the benefit of U.S. Provisional Application No. 63/292,521, filed Dec. 22, 2021, which is incorporated by reference herein in its entirety.

### TECHNICAL FIELD

The present disclosure relates generally to calcium arc measurement in intravascular imaging. In particular, a calcium arc is automatically calculated and displayed for an intravascular image in response to a user input selecting two locations on the image.

### BACKGROUND

Physicians use many different medical systems and tools to monitor a patient's health and diagnose and treat medical conditions. Different modalities of medical diagnostic systems may provide a physician with different images, models, and/or data relating to internal structures within a patient. These modalities include invasive devices and systems, such as intravascular systems, and non-invasive devices and systems, such as x-ray systems, and computed tomography (CT) systems. Treatment procedures also include invasive and non-invasive treatment devices and systems designed to increase or restore blood flow in constricted blood vessels.

In the field of intravascular ultrasound (IVUS) imaging, a physician may view and compare images of the interior of a blood vessel to determine locations which may need treatment. To facilitate accurate and efficient comparison of IVUS images, the extent of obstructive material, such as calcium, may be quantified. Specifically, a calcium arc measurement may refer to an angle of a circular IVUS image which shows calcium deposits within that region of the image. Knowing a calcium arc is an important part of determining if vessel preparation is required prior to stenting a coronary lesion.

Currently, physicians may determine calcium arc measurements manually by measuring regions of an IVUS image or be visual estimation. Manual calculation of calcium arc measurements may lead to a greater risk of error and could slow the procedure time of an imaging procedure or another diagnostic or treatment procedure.

### SUMMARY

Embodiments of the present disclosure are systems, devices, and methods for automatically calculating a calcium arc measurement in response to a user selecting locations within an intravascular image (e.g., an intravascular ultrasound or IVUS image, an optical coherence tomography or OCT image). Aspects of the present disclosure advantageously provide a physician with a tool to quickly identify regions of calcium deposits within an intravascular image. Features of the present disclosure advantageously assist a physician to make accurate calcium arc measurements as well as reduce procedure time for an IVUS imaging or analysis procedure.

In one aspect, an intravascular imaging device is placed in a blood vessel of a patient. As the device is moved through the vessel, multiple intravascular images are obtained. As one intravascular image is displayed to a user, the user may select two locations within the image. The user may select two locations by touching a location on a touch-screen input and display device, dragging their finger to a different location on the device, and lifting their finger from the device. The location at which the user first touched the device defines the first location, and the location at which the user removed their finger from the device defines the second location. A processor circuit then calculates an angle between the first and second locations and displays the angle as the calcium arc. In some aspects, multiple angles of calcium arc measurements are calculated and displayed for the same intravascular image.

According to an exemplary aspect, a system is provided. The system includes a processor circuit configured for communication with an intraluminal imaging device and an input device, wherein the processor circuit is configured to: receive an intraluminal image obtained by the intraluminal imaging device while the intraluminal imaging device is positioned within a body lumen of a patient; receive, via the input device, a first user input selecting a first location within the intraluminal image; receive, via the input device, a second user input selecting a second location within the intraluminal image, wherein the first location and the second location define a boundary associated with a first occurrence of a tissue type depicted in the intraluminal image; determine a first angle between the first location and the second location; output, to a display in communication with the processor circuit, a screen display comprising: the intraluminal image; and a visual representation of the first angle.

In some aspects, the visual representation of the first angle comprises a numerical value of the first angle. In some aspects, the visual representation of the first angle comprises an overlay visually distinguishing a first portion of the intraluminal image within the boundary from a second portion of the intraluminal image outside the boundary. In some aspects, the intraluminal image comprises a circumferential image, and the visual representation of the first angle comprises an arc-shaped portion of the circumferential image. In some aspects, the input device comprises a touch input device, and the first user input comprises a start of a touch input and the second user input comprises an end of the touch input. In some aspects, the touch input comprises a line segment within the intraluminal image, and the angle is representative of an arc-shaped portion of the intraluminal image. In some aspects, the screen display further comprises: a first indicator positioned relative to the intraluminal image to identify the first location; and a second indicator positioned relative to the intraluminal image to identify the second location. In some aspects, the processor circuit is further configured to: receive, by the input device, a third user input changing the second location to be a third location within the intraluminal image; calculate a second angle between the first location and the third location; and change the screen display to include a visual representation of the angle and to remove the visual representation of the first angle. In some aspects, the third user input comprises a movement of a handle in the screen display from the second location to the third location. In some aspects, the screen display further comprises: a first handle corresponding to the first location and a second handle corresponding to the second location. In some aspects, the processor circuit is further configured to: receive, by the input device, a third user input selecting a third location within the intraluminal image; receive, by the input device, a fourth user input selecting a fourth location within the intraluminal image, wherein the third location and the fourth location define a boundary associated with a second occurrence of the tissue type depicted in the intraluminal image; and determine a second angle between the third location and the fourth location; wherein the screen display comprises a visual representation of the second angle simultaneously as the visual representation of the first angle. In some aspects, the processor circuit is configured to calculate a total angle for the tissue type based on a sum of the first angle and the second angle, and the screen display comprises a visual representation of the total angle. In some aspects, the body lumen comprises a blood vessel, and the tissue type comprises calcium within a wall of the blood vessel.

According to an exemplary aspect, a method is provided. The method includes receiving, with a processor circuit in communication with an intraluminal imaging device, an intraluminal image obtained by the intraluminal imaging device while the intraluminal imaging device is positioned within a body lumen of a patient; receiving, with the processor circuit, a first user input via an input device in communication with the processor circuit, wherein the first user input selects a first location within the intraluminal image; receiving, with the processor circuit, a second user input via the input device, wherein the second user input selects a second location within the intraluminal image, wherein the first location and the second location define a boundary associated with a first occurrence of a tissue type depicted in the intraluminal image; determining, with the processor circuit, an angle between the first location and the second location; and outputting, to a display in communication with the processor circuit, a screen display comprising: the intraluminal image; and a visual representation of the angle.

According to an exemplary aspect, a system is provided. The system includes an intravascular imaging device; and a processor circuit configured for communication with the intraluminal imaging device and an input device, wherein the processor circuit is configured to: receive an intravascular image obtained by the intravascular imaging device while the intravascular imaging device is positioned within a blood vessel of a patient; receive, via the input device, a first user input selecting a first location within the intravascular image; receive, via the input device, a second user input selecting a second location within the intravascular image, wherein the first location and the second location define a boundary associated with an occurrence of a tissue type depicted in the intravascular image; determine an angle between the first location and the second location; output, to a display in communication with the processor circuit, a screen display comprising: the intravascular image; and a visual representation of the angle.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 is a schematic diagram of a processor circuit, according to aspects of the present disclosure.

FIG. 3 is a diagrammatic view of a graphical user interface displaying a selected view of an IVUS image, according to aspects of the present disclosure.

FIG. 4 is a diagrammatic view of a graphical user interface displaying a selected view of an IVUS image, according to aspects of the present disclosure.

FIG. 5 is a diagrammatic view of a graphical user interface displaying a selected view of an IVUS image and a calcium arc measurement, according to aspects of the present disclosure.

FIG. 6 is a diagrammatic view of a graphical user interface displaying selected views of an IVUS image and calcium arc measurements, according to aspects of the present disclosure.

FIG. 7 is a diagrammatic view of a graphical user interface displaying selected views of an IVUS image and calcium arc measurements, according to aspects of the present disclosure.

FIG. 8 is a flow diagram of a method of automatically adjusting laser atherectomy settings based on coregistration of intraluminal data and extraluminal data, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

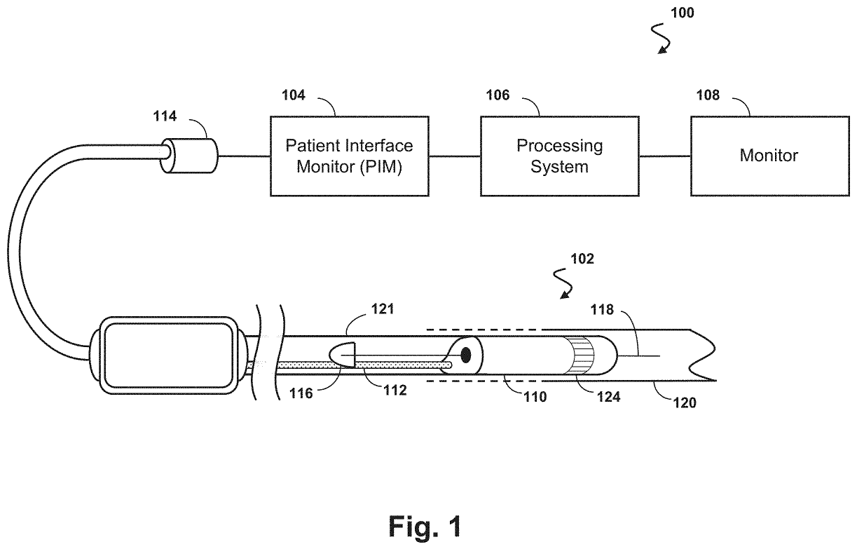

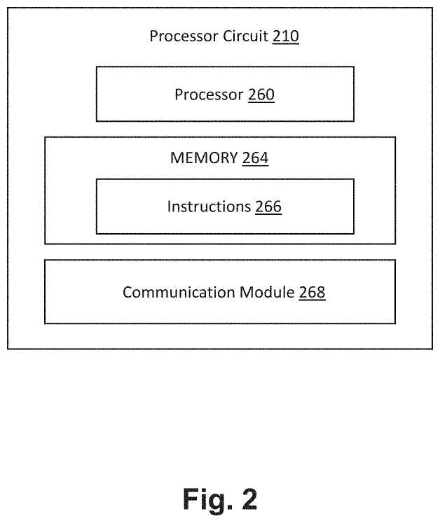

FIG. 1 is a diagrammatic schematic view of an intraluminal imaging system 100, according to aspects of the present disclosure. The system 100 may be a system comprising a processor circuit configured for communication with an intravascular imaging catheter 102 and a display 108. The system 100 may be a system comprising a processor circuit configured for communication with an intravascular ultrasound (IVUS) imaging catheter 102 and a display 108. The processor circuit may be the processor circuit 210 described with reference to FIG. 2. The display 108 may also be referred to as a monitor, as shown in FIG. 1. The intraluminal imaging system 100 can be an ultrasound imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, an processing system or console 106, and a monitor 108. The intraluminal imaging device 102 can be an ultrasound imaging device. In some instances, the device 102 can be an IVUS imaging device, such as a solid-state IVUS device. The intraluminal imaging device 102 may also be referred to as an intraluminal imaging catheter. The intraluminal imaging device may also be referred to as an intravascular ultrasound (IVUS) imaging catheter.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

The PIM 104 facilitates communication of signals between the IVUS console 106 and the scanner assembly 110 included in the IVUS device 102. This communication includes the steps of: (1) providing commands to integrated circuit controller chip(s) 206A and 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A and 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s)126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the transmission line bundle or cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

It is understood that the system 100 and/or device 102 can be configured to obtain any suitable intraluminal imaging data. In some embodiments, the device 102 may include an imaging component of any suitable imaging modality, such as optical coherence tomography (OCT), intracardiac echocardiography (ICE), etc.

FIG. 2 is a schematic diagram of a processor circuit 210, according to aspects of the present disclosure. The processor circuit 210 may be implemented in the processing system 106 of FIG. 1. In an example, the processor circuit 210 may be in communication with the intraluminal imaging device 102, the x-ray imaging system 109, and/or the display 108 within the system 100. The processor circuit 210 may include a processor and/or communication interface. One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to the probe 110 and/or the processing system 106 (FIG. 1). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the probe 110, and/or the display or monitor 108. In that regard, the communication module 268 can be an input/output (I/O) device. In some instances, the communication module 268 facilitates direct or indirect communication between various elements of the processor circuit 210 and/or the probe 110 (FIG. 1) and/or the processing system 106 (FIG. 1).

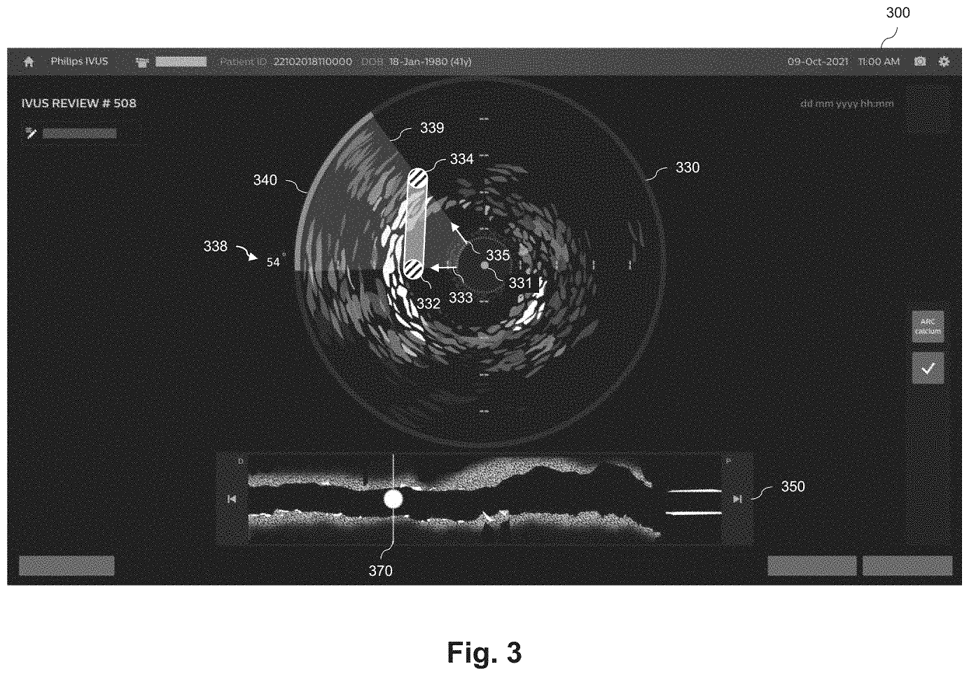

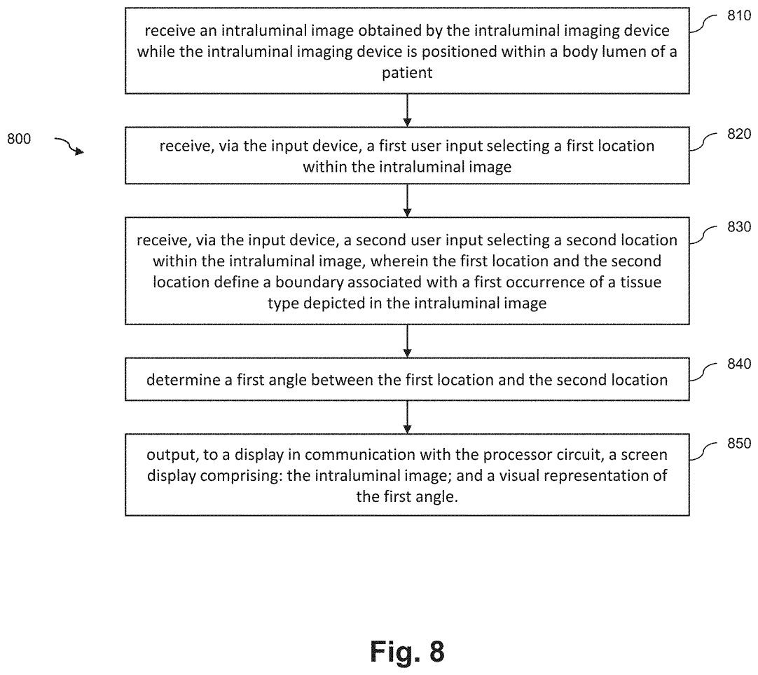

FIG. 3 is a diagrammatic view of a graphical user 300 interface displaying a selected view of an IVUS image 330, according to aspects of the present disclosure. FIG. 3 illustrates an exemplary graphical user interface 300, according to aspects of the present disclosure. The interface 300 may include an IVUS image 330 and an ILD 350. In some aspects, the IVUS image 330 may by an intraluminal image of any suitable type. For example, any of the principles described herein, including any of the steps of the method 800 describes with reference to FIG. 8, or any other teaching, may be applied to an OCT image acquired during an OCT imaging procedure.

In some embodiments, the processor circuit 210 (FIG. 2) may be configured to acquire intravascular ultrasound (IVUS) data during an IVUS pullback imaging procedure, as described with reference to FIG. 1. The processor circuit 210 may generate multiple IVUS images, like the image 330 shown, based on this received IVUS data. Each IVUS image received may include a radial cross-sectional view of a vessel of the patient. In some cases, a medical treatment device, such as a stent, may be present within the vessel imaged by the intravascular imaging system.

The graphical user interface 300 may include a longitudinal intraluminal image 350. The longitudinal image 350 can be referred to as in-line digital (ILD) display or image longitudinal display (ILD). The IVUS images acquired during an intravascular ultrasound imaging procedure, such as during an IVUS pullback, may be used to create the ILD 350. In that regard, an IVUS image is a tomographic or radial cross-sectional view of the blood vessel. The ILD 350 provides a longitudinal cross-sectional view of the blood vessel. The ILD 350 can be a stack of the IVUS images acquired at various positions along the vessel, such that the longitudinal view of the ILD 350 is perpendicular to the radial cross-sectional view of the IVUS images. In such an embodiment, the ILD 350 may show the length of the vessel, whereas an individual IVUS image is a single radial cross-sectional image at a given location along the length. In another embodiment, the ILD 350 may be a stack of the IVUS images acquired overtime during the imaging procedure and the length of the ILD 350 may represent time or duration of the imaging procedure. The ILD 350 may be generated and displayed in real time or near real time during the pullback procedure. As each additional IVUS image is acquired, it may be added to the ILD 350. For example, at a point in time during the pullback procedure, the ILD 350 shown in FIG. 3 may be partially complete. In some embodiments, the processor circuit may generate an illustration of a longitudinal view of the vessel being imaged based on the received IVUS images. For example, rather than displaying actual vessel image data as the ILD 350 does, the illustration may be a stylized version of the vessel, with e.g., continuous lines showing the lumen border, a stent border, and/or the vessel border.

The ILD 350 may include a depiction of the vessel. The ILD 350 may additionally include an indicator 370. The indicator 370 may represent the location at which the IVUS image 330 displayed was acquired. In some embodiments, the processor circuit 210 may be configured to receive an input from the user. The user input may select and move the indicator 370 to a new location along the ILD 350. The user input may alternatively select a location along the ILD 350. In response to either of these user inputs, the processor circuit 210 may be configured to display a new IVUS image associated with the new selected location along the ILD 350.

In some embodiments, the processor circuit 210 may be configured to receive an input from the user selecting one or more locations on the IVUS image 330 displayed. For example, in some embodiments, the user may select two locations, a location 332 and a location 334 on the image 330. In some examples, the user input can be a single tap on a touchscreen device or a click with a mouse or any other input of an input device. In some examples, the user input may be a swipe. For example, a swipe may include touching a location of a touch screen with a user's finger and moving across the touchscreen to a second location. Other methods of receiving a user input may also be used. The locations 332 and 334 may form a line segment between the two locations. As shown in FIG. 3, this line segment may be displayed within the interface 300. In some examples, the location 332 and the location 334, and/or the line segment between the location 332 and the location 334, may correspond to a sector, or arc-shaped portion, of the image 330 showing calcium within the image 330. For example, the location 334 and 331 may define an outer scan line of a sector of the image 332 and the location 331 may define a second outer scan line of the sector.

In some embodiments, the arc measurement is generated directly in response to the user input specifying the location of the arc (e.g., a touch-drag-lift input from 334 to 332 or vice versa). In some embodiments, the user enters a calcium arc measurement mode (e.g., by providing a separate user input to enter a calcium arc measurement mode). In some embodiments, a button or other user interface element may be provided allowing a user to input a direction to enter a calcium arc measurement mode. In other embodiments, the user does not need to provide a user input to enter a separate calcium arc measurement mode. For example, the user may provide the touch input specifying the extent of the arc (e.g., a touch-drag-lift from the location 332 to the location 334 or vice versa). This input may direct the processor circuit 510 of the system to measure the calcium arc.

In some embodiments, the indicator 334 indicates where a touch input began (e.g., putting a finger on a touchscreen), the path between the indicators 334 and 332 may correspond to where the touch input continued (e.g., dragging a finger across a touch screen), and the indicator 332 may correspond to where the touch input ended (e.g., lifting a finger).

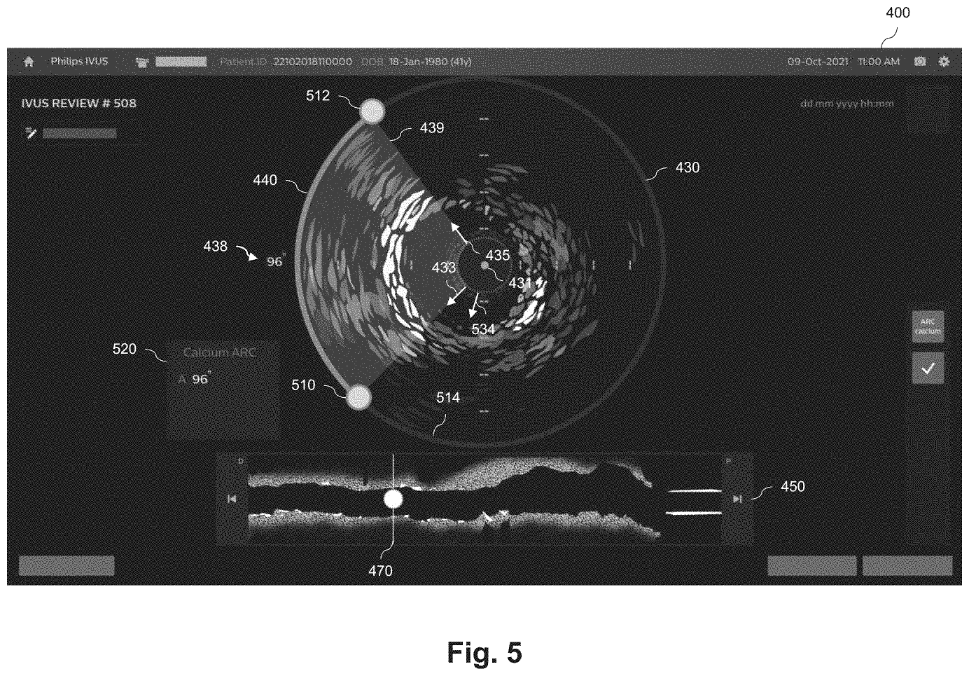

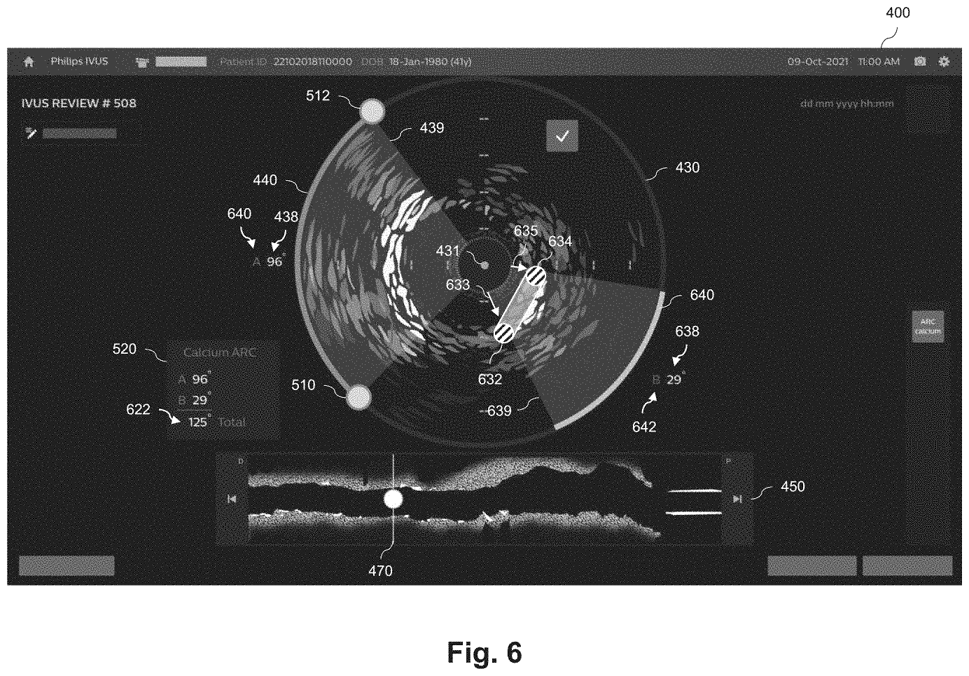

In some embodiments, an arc shaped measurement, such as a calcium arc measurement, can be performed based on a user input that is differently shaped from the one illustrated by the path between the indicators 332 and 334. In particular, the touch input may not be linear but may be of any suitable shape. This may advantageously simplify how the user measures the arc by allowing for any suitable path between two points. In the illustrated embodiments shown in FIG. 3, the path of the touch input is vertical. However, it could also be horizontal or a combination of vertical and horizontal (e.g., diagonal as shown and described in FIG. 6). In some embodiments, the touch input could also be arc shaped (e.g., a press-touch-drag of one of the anchors 510, 512 around the circumference as will be described in more detail with reference to FIG. 5). The touch input could also be arc shaped within the image (e.g., the pathway between 332 and 334).

In some embodiments, the processor circuit 210 may be configured to determine an angle 333 associated with the location 332. The angle 333 may refer to a direction extending from a center point 331 of the image 330 to an outer edge of the image 330. In one embodiment, the direction of a scan line may be defined by a line drawn between the location 332 and the center point 331 of the image 330. The angle 333 may refer to a direction of that scan line. For example, the angle 333 may be defined as an angle between the scan line defined by the location 332 and the center point 331 and a scan line extending from the center 331 to another reference point within the image 330. For example, an angle of zero degrees may be defined as a scanline extending from the central point 331 to an uppermost point along the circumference of the image 330, or any other reference point within the image 330.

Similarly, an angle 335 may be calculated corresponding to the location 334 selected by the user. The angle 335 may be calculated according to a similar method as described with reference to the angle 333. The processor circuit 210 may be configured to determine a difference between the angle 333 and the angle 335. This difference in angles may correspond to an angle 338. This angle 338 may be displayed within the graphical user interface 300. The angle 338 may be displayed proximate to the image 330 and/or the sector 339. In an embodiment in which the user of the system 100 selects the locations 332 and 334 corresponding to the presence of calcium in the image 330, the angle 338 may be referred to as a calcium arc.

In some embodiments, the locations 332 and 334 and/or the angles 333 and 335 define a sector 339 of the image 330. The sector 339 may alternatively be referred to as an arc, circumferential segment, or any other suitable term. The sector 339 may correspond to regions of the image 330 showing the presence of calcium. In some embodiments, the sector 339 may be differentiated from other regions of the image 330 by the processor circuit 210. For example, the sector 339 may be differentiated by highlighting or shading regions of the image 330 corresponding to the sector 339. In addition, in some embodiments, a line 340 may be placed along the circumference of the image 330 at the sector 339. Other methods of differentiation of the sector 339 are also contemplated. For example, the processor circuit 210 may differentiate the sector 339 from other regions of the image 330 by using various visual elements such as various patterns, colors, indicators, or any other visual elements.

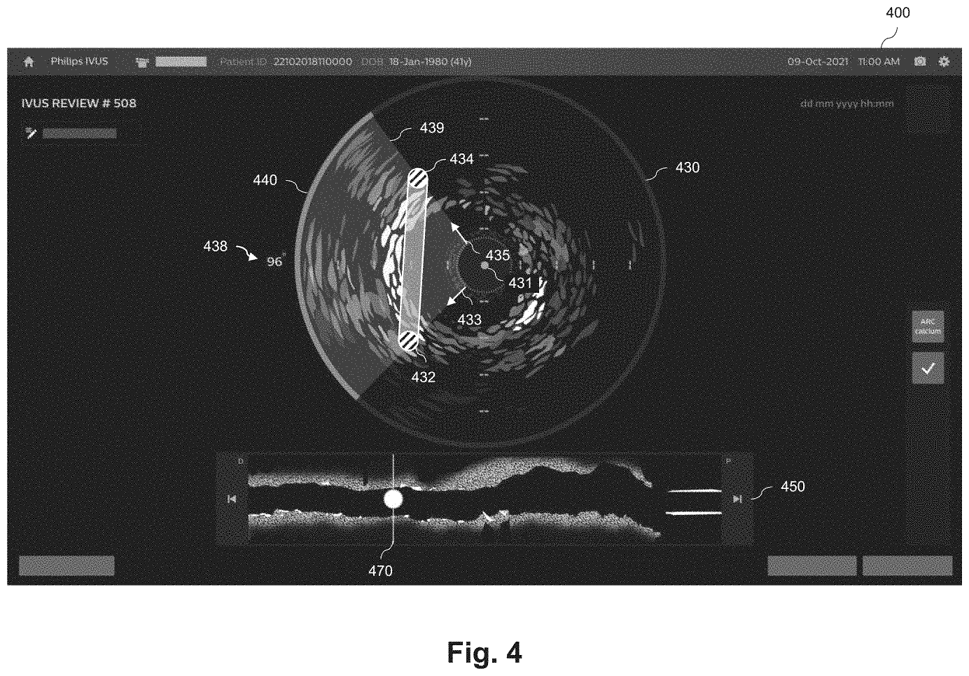

FIG. 4 is a diagrammatic view of a graphical user interface 400 displaying a selected view of an IVUS image 430, according to aspects of the present disclosure. FIG. 4 illustrates an exemplary graphical user interface 400, according to aspects of the present disclosure. The interface 400 may be similar to the graphical user interface 300. For example, the interface 400 may include a display of an IVUS image 430 and a corresponding ILD 450 after a user of the system 100 has selected a location 432 and a location 434 with a user input device. In some aspects, the input device and the display device (e.g., monitor 108) may be the same component or integrated in a single housing (e.g., a touchscreen display/monitor).

As previously described with reference to FIG. 3, the graphical user interface 400 may depict locations 432 and 434 selected by the user. The interface 400 may also depict angles 433 and 435 respectively corresponding to these locations. The processor circuit 210 may define the direction associated with the angle 433 by a line between the location 432 and the center point 431. The angle 435 may be similarly defined. A difference between the angles 433 and 435 may be displayed as the angle 438. This angle may be a calcium arc. The locations 432 and 434 may together define a sector 439. A line 440 may further identify the region associated with the sector 439. The ILD 450 may display a longitudinal view of the vessel and may include an indicator 470 identifying the location along the vessel at which the image 430 was obtained.

FIG. 5 is a diagrammatic view of a graphical user interface 400 displaying a selected view of an IVUS image 430 and a calcium arc measurement 520, according to aspects of the present disclosure. FIG. 5 illustrates the graphical user interface 400 after a user has selected locations corresponding to a calcium arc, according to aspects of the present disclosure. After a user selects locations (e.g., the locations 432 and 434 of FIG. 4), the processor circuit 210 may display anchors 510 and 512. In some embodiments, the anchors 510 and 512 may also be referred to as handles. The anchors 510 and 512 may be positioned at either end of the line 440 along the circumference of the image 430. In this aspect, the anchors 510 and 512 may define the sector 439 within the image 430. The anchors 510 and 512 may be positioned at other locations. For example, they may be placed at any locations along the outermost scanlines of the sector 439 as shown by the angles 433 and 435.

In some embodiments, the processor circuit 210 may be configured to display metrics 520. The metrics 520 may include any suitable measurements or data. In one example, the metrics 520 may include the angle or calcium arc measurement 438 corresponding to the sector 439. In some embodiments, the sector 439 may be associated with a label. As shown in FIG. 5, an exemplary label “A” may be assigned to the sector 439. This label, along with the calcium arc measurement 438 may be included as metrics 520. In some embodiments, the user of the system may rename any labels associated with calcium arcs by a user input.

In some embodiments, the processor circuit 210 may be configured to receive an additional user input. A user input may include the selection of one of the anchors 510 and/or 512. A user may select one of these anchors 510 or 512 and move them to another location along the circumference of the image 430. The anchors 510 and 512 may be adjusted to more accurately correspond to the locations of calcium within the image 430. For example, the anchors 510 and/or 512 may be adjusted such that the sector 439 may include sections of the image 430 which also include calcium deposits. In one example, the anchor 510 may be moved to the location 514 of the image 430 in response to a user input. After the anchor 510 is moved to the location 514, the processor circuit may calculate a new angle 534 associated with the new position of the anchor 510. For example, the processor circuit may determine this angle as a line between the location 514 and the center point 431 of the image 430. This angle may be compared with the angle 435 of anchor 512 to update the angle 438 to reflect the change. The sector 439 and line 440 may also be updated to reflect the change. The metrics 520 may also be updated to reflect the change.

FIG. 6 is a diagrammatic view of a graphical user interface 400 displaying selected views of an IVUS image 430 and calcium arc measurements 520, according to aspects of the present disclosure. FIG. 6 illustrates the graphical user interface 400 after a user has selected two additional locations, according to aspects of the present disclosure. As shown in the example in FIG. 6, the processor circuit 210 may be configured to receive additional inputs associated with other sectors of the image 430 corresponding to additional plaque presence.

As shown in FIG. 6, after the sector 439 is identified by the user and the associated calcium arc 438 is calculated and displayed, the user may select additional locations within the image 430, such as locations 632 and 634. In some embodiments, these locations 632 and 634 may identify a region of the image 430 showing additional calcium deposits. The locations 632 and 634 may be selected by any suitable method of input, including any of those described with reference to locations 332 and 334 of FIG. 3.

Similar to the calculation of angles 433 and 435, the angles 633 and 635 corresponding to the locations 632 and 634 respectively may be calculated. The angles 633 and 635 may calculated by any suitable method, including those described with reference to the calculation of the angles 333 and 335 of FIG. 3 described previously. For example, a line between the location 632 and the center point 431 may be determined and an angle relative to a reference scan line may be determined. A similar procedure may be followed to determine the angle 635.

A sector 639 may be defined by the locations 632 and 634. The sector 639 may identify the locations within the image 430 showing a presence of calcium. In some embodiments, a line 640 may also be displayed along the circumference of the image 430 and corresponding to the locations of the sector 639.

A difference between the angles 632 and 634 may be determined and displayed as the angle 638. The angle 638 may be displayed proximate to the image 430 and the sector 639. In an embodiment in which multiple sectors of the image are identified by the user, each sector may be assigned a unique label. This label may be determined and assigned by the processor circuit 210 according to any suitable order, or may be determined and assigned by the user of the system 100. In the example shown in FIG. 6, the sector 439 and its accompanying calcium arc measurement 438 may be assigned the label 640 “A.” Similarly, the sector 639 and its accompanying calcium arc measurement 638 may be assigned the label 642 “B.”

In some embodiments, after an additional sector, such as the sector 639 is identified and displayed, the metrics 520 may be updated. For example, the metrics 520 may include the labels associated with all identified sectors of the image. As shown in FIG. 6, the label 640 and measurement 438 may be included first in the metrics 520 and the label 642 and measurement 638 may be displayed as well. In some embodiments in which multiple sectors showing calcium are identified within an image 430, the processor circuit 210 may be configured to calculate a sum of all calcium arc calculations. This sum 622 may be included in the metrics 520. In some embodiments, the sum 622 may advantageously provide a user of the system with a total amount of degrees of the circular image 430 corresponding to the presence of calcium. This metric may assist the user or a physician in determining proper treatment therapies to restore blood flow of the vessel or prepare the vessel for a procedure. This metric may be a numerical value, a visual representation, or any other graphical element.

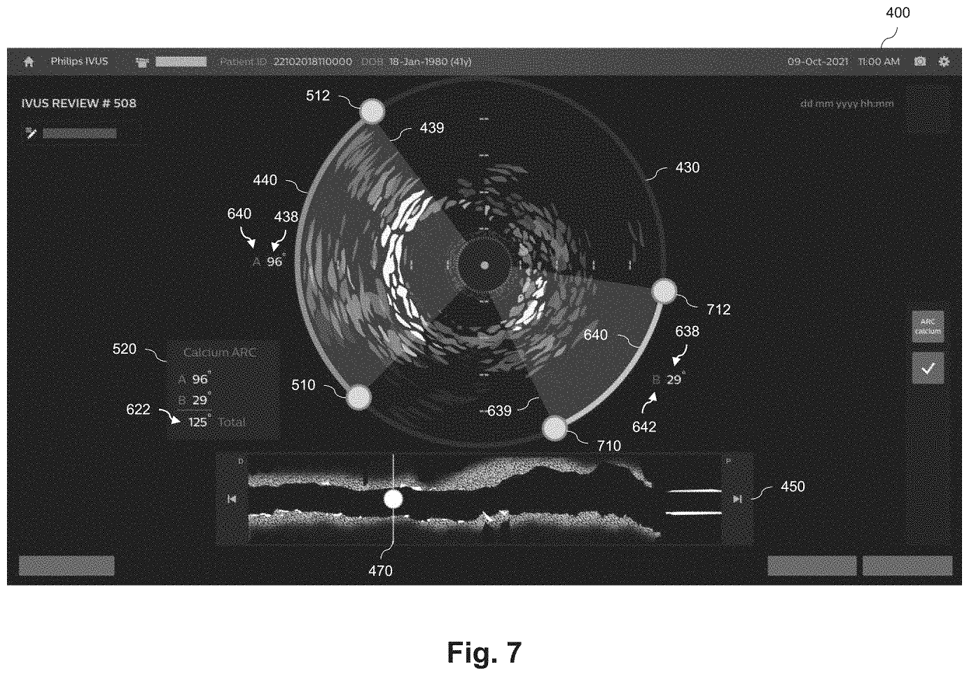

FIG. 7 is a diagrammatic view of a graphical user interface 400 displaying selected views of an IVUS image 430 and calcium arc measurements, according to aspects of the present disclosure. FIG. 7 illustrates the graphical user interface 400 after a user has selected additional locations within the image 430, according to aspects of the present disclosure. After a user selects locations (e.g., the locations 632 and 634 of FIG. 6) for an additional sector (e.g., the sector 639), the processor circuit 210 may display anchors 710 and 712. The anchors 710 and 712 may be similar to the anchors 510 and 512 described with reference to FIG. 5. In some embodiments, the anchors 710 and 712 may also be referred to as handles. The anchors 710 and 712 may be positioned at either end of the line 640 along the circumference of the image 430.

In some embodiments, the processor circuit 210 may be configured to receive an additional user input adjusting the locations of the anchors 710 and/or 712. A user input may include the selection of one of the anchors 710 and/or 712. A user may select one of these anchors 710 or 712 and move them to another location along the circumference of the image 430. The anchors 710 and 712 may be adjusted to more accurately correspond to the locations of calcium within the image 430. After either of the anchors 710 or 712 are moved, the processor circuit may be configured to determine a new angle associated with the new location. The angle 638 may then be updated based on the new location of one of the anchors 710 or 712. The sector 639 and line 640 may also be updated to reflect the change. The metrics 520, including the sum 622, may also be updated to reflect the change.

In some embodiments, aspects of the present disclosure may include a coregistration process. For example, a coregistration process may include performing an IVUS imaging pullback procedure while also acquiring extraluminal images, such as no-contrast x-ray images, including fluoroscopy images and/or cine images. In particular, during an IVUS imaging procedure, a physician may position an IVUS imaging device (e.g., the device 102 of FIG. 1) at a distal position within a vessel. After or before the IVUS imaging device is positioned at this distal location, the physician may direct an extraluminal imaging system, such as an x-ray system, to continuously obtain extraluminal images of the patient anatomy such that the extraluminal images show a radiopaque portion of the IVUS imaging device in the vessel. The physician may then direct the IVUS imaging device to begin to acquire IVUS images. In that regard, both the IVUS imaging device and the extraluminal imaging device may receive respective images simultaneously. After the pullback is performed, the system may identify the location of the radiopaque marker of the IVUS imaging device within each acquired extraluminal image. In this way, each extraluminal image may be associated with at least one position of the IVUS device. These positions, which may be stored as coordinates of pixels within the extraluminal image, may define a pathway of the IVUS imaging device. Each location along this pathway may be associated with one or more IVUS images obtained by the IVUS imaging device at that location along the pathway as seen in an extraluminal image. The IVUS imaging device pathway may then be overlaid over an extraluminal image, such as one of the extraluminal images obtained previously, or a separate extraluminal image (e.g., a contrast-filled angiogram image). As a result, in some embodiments, a user of the system may advantageously be able to see a location within an extraluminal image of the vessel associated with an IVUS image obtained at that location. Based on this same location data, the scale 352 shown associated with the ILD 350 may be based on locations (e.g., in mm) of IVUS images received. In this way, data associated with an IVUS image, including calcium arc data, such as the data 520, a sum 622, the positions of sectors 439 and/or 639, or any other data may be coregistered to an extraluminal image. These data may be displayed adjacent to regions of an imaged vessel within an extraluminal image.

FIG. 8 is a flow diagram of a method 800 of automatically adjusting laser atherectomy settings based on coregistration of intraluminal data and extraluminal data, according to aspects of the present disclosure. The method 800 may describe an automatic segmentation of a vessel to detect segments of interest using co-registration of invasive physiology and x-ray images. As illustrated, the method 800 includes a number of enumerated steps, but embodiments of the method 800 may include additional steps before, after, or in between the enumerated steps. In some embodiments, one or more of the enumerated steps may be omitted, performed in a different order, or performed concurrently. The steps of the method 800 can be carried out by any suitable component within the system 100 and all steps need not be carried out by the same component. In some embodiments, one or more steps of the method 800 can be performed by, or at the direction of, a processor circuit of the diagnostic system 100, including, e.g., the processor 260 (FIG. 2) or any other component.

At step 810, the method 800 includes receiving an intraluminal image obtained by the intraluminal imaging device while the intraluminal imaging device is positioned within a body lumen of a patient. In some aspects, the method 800 may include receiving an intravascular image obtained by the intravascular imaging device while the intravascular imaging device is positioned within a blood vessel of a patient. In some aspects, the first location may be within the intraluminal image or the intravascular image. In some aspects, the first location could be along the border of the intraluminal image or the intravascular image. In some aspects, the location could be outside the intraluminal image or the intravascular image and correspond to a boundary line extending from the center of the intraluminal image or intravascular image to the first location.

At step 820, the method 800 includes receiving, via the input device, a first user input selecting a first location within the intraluminal image. In some aspects, the method 800 may include receiving, via the input device, a first user input selecting a first location within the intravascular image.

At step 830, the method 800 includes receiving, via the input device, a second user input selecting a second location within the intraluminal image, wherein the first location and the second location define a boundary associated with a first occurrence of a tissue type depicted in the intraluminal image. In some aspects, the method 800 may include receiving, via the input device, a second user input selecting a second location within the intravascular image, wherein the first location and the second location define a boundary associated with an occurrence of a tissue type depicted in the intravascular image.

At step 840, the method 800 includes determining a first angle between the first location and the second location. In some aspects, the method 800 includes determining an angle between the first location and the second location.

At step 850, the method 800 includes outputting, to a display in communication with the processor circuit, a screen display comprising: the intraluminal image; and a visual representation of the first angle. In some aspects, the method 800 includes outputting, to a display in communication with the processor circuit, a screen display comprising: the intravascular image; and a visual representation of the angle.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an intraluminal imaging device and an input device, wherein the processor circuit is configured to:
receive an intraluminal image obtained by the intraluminal imaging device while the intraluminal imaging device is positioned within a body lumen of a patient;
receive, via the input device, a first user input selecting a first location within the intraluminal image;
receive, via the input device, a second user input selecting a second location within the intraluminal image, wherein the first location and the second location define a boundary associated with a first occurrence of a tissue type depicted in the intraluminal image;
determine a first angle between the first location and the second location;
output, to a display in communication with the processor circuit, a screen display comprising:
the intraluminal image; and
a visual representation of the first angle.

2. The system of claim 1, wherein the visual representation of the first angle comprises a numerical value of the first angle.

3. The system of claim 1, wherein the visual representation of the first angle comprises an overlay visually distinguishing a first portion of the intraluminal image within the boundary from a second portion of the intraluminal image outside the boundary.

4. The system of claim 1,
wherein the intraluminal image comprises a circumferential image, and
wherein the visual representation of the first angle comprises an arc-shaped portion of the circumferential image.

5. The system of claim 1,
wherein the input device comprises a touch input device, and
wherein the first user input comprises a start of a touch input and the second user input comprises an end of the touch input.

6. The system of claim 5,
wherein the touch input comprises a line segment within the intraluminal image, and
wherein the angle is representative of an arc-shaped portion of the intraluminal image.

7. The system of claim 1, wherein the screen display further comprises:
a first indicator positioned relative to the intraluminal image to identify the first location; and
a second indicator positioned relative to the intraluminal image to identify the second location.

8. The system of claim 1, wherein the processor circuit is further configured to:
receive, by the input device, a third user input changing the second location to be a third location within the intraluminal image;
calculate a second angle between the first location and the third location; and
change the screen display to include a visual representation of the angle and to remove the visual representation of the first angle.

9. The system of claim 8, wherein the third user input comprises a movement of a handle in the screen display from the second location to the third location.

10. The system of claim 1, wherein the screen display further comprises:
a first handle corresponding to the first location; and
a second handle corresponding to the second location.

11. The system of claim 1,
wherein the processor circuit is further configured to:
receive, by the input device, a third user input selecting a third location within the intraluminal image;
receive, by the input device, a fourth user input selecting a fourth location within the intraluminal image, wherein the third location and the fourth location define a boundary associated with a second occurrence of the tissue type depicted in the intraluminal image; and
determine a second angle between the third location and the fourth location;
wherein the screen display comprises a visual representation of the second angle simultaneously as the visual representation of the first angle.

12. The system of claim 11,
wherein the processor circuit is configured to calculate a total angle for the tissue type based on a sum of the first angle and the second angle, and
wherein the screen display comprises a visual representation of the total angle.

13. The system of claim 1,
wherein the body lumen comprises a blood vessel, and
wherein the tissue type comprises calcium within a wall of the blood vessel.

14. A method, comprising:
receiving, with a processor circuit in communication with an intraluminal imaging device, an intraluminal image obtained by the intraluminal imaging device while the intraluminal imaging device is positioned within a body lumen of a patient;
receiving, with the processor circuit, a first user input via an input device in communication with the processor circuit, wherein the first user input selects a first location within the intraluminal image;
receiving, with the processor circuit, a second user input via the input device, wherein the second user input selects a second location within the intraluminal image, wherein the first location and the second location define a boundary associated with a first occurrence of a tissue type depicted in the intraluminal image;
determining, with the processor circuit, an angle between the first location and the second location; and
outputting, to a display in communication with the processor circuit, a screen display comprising:
the intraluminal image; and
a visual representation of the angle.

15. A system, comprising:
an intravascular imaging device; and
a processor circuit configured for communication with the intraluminal imaging device and an input device, wherein the processor circuit is configured to:
receive an intravascular image obtained by the intravascular imaging device while the intravascular imaging device is positioned within a blood vessel of a patient;
receive, via the input device, a first user input selecting a first location within the intravascular image;
receive, via the input device, a second user input selecting a second location within the intravascular image, wherein the first location and the second location define a boundary associated with an occurrence of a tissue type depicted in the intravascular image;
determine an angle between the first location and the second location;
output, to a display in communication with the processor circuit, a screen display comprising:
the intravascular image; and
a visual representation of the angle.
